(19) United States
(12) Patent Application Publication
(10) Pub. No.: US 2026/0215790 A1
(43) Pub. Date: Jul. 30, 2026

(54) SURGICAL INSTRUMENT FOR CONDITIONING LARGE DIAMETER BURR HOLES

(71) Applicant: The Regents of the University of Colorado, A Body Corporate, Denver, CO (US)

(72) Inventors: Joshua Seinfeld, Denver, CO (US); Zach Folzenlogen, Denver, CO (US); Michael Bojanowski, Denver, CO (US); Joshua Grip, Arvada, CO (US); Richard Wojcik, Denver, CO (US)

(73) Assignee: The Regents of the University of Colorado, A Body Corporate, Denver, CO (US)

(21) Appl. No.: 19/143,905

(22) PCT Filed: Jan. 5, 2024
(86) PCT No.: PCT/US2024/010455

§ 371 Date: Jun. 27, 2025

Related U.S. Application Data
(60) Provisional application No. 63/478,779, filed on Jan. 6, 2023.

(51) Int. Cl. A61B 17/16; A61B 90/00

(52) U.S. Cl. CPC A61B 17/1615; A61B 17/1633; A61B 17/1695; A61B 90/06; A61B 90/08; A61B 2090/062; A61B 2090/0801

(57) ABSTRACT

Devices and methods for evacuation of subdural hematomas, with a reduced recurrence rate, such that the use of general anesthesia can be avoided, and subdural hematomas can be treated with reduced risk to the impacted population. The devices include a precision cutting tool that is used to create a uniform diameter within a pre-existing hole in a target area of a subject, thereby maximizing the diameter-to-depth ratio of the hole, while simultaneously removing materials both radially and axially. The methods include conditioning an initial hole to facilitate the installation of a port within an implantation hole in a target area of a subject.

### CROSS-REFERENCE TO RELATED APPLICATIONS

This nonprovisional application claims priority to U.S. provisional application No. 63/478,779, entitled “SYSTEMS AND DEVICES FOR LARGE BURR HOLE INTRACRANIAL ACCESS AND EVACUATION,” filed Jan. 6, 2023 by the same inventors.

### BACKGROUND OF THE INVENTION

### 1. Field of the Invention

This invention relates, generally, to systems, devices, and methods to provide intracranial access. More specifically, it relates to devices and methods to create large diameter burr holes having consistent diameters throughout to enable installation of a large diameter port and improve surgical procedural outcomes.

### 2. Brief Description of the Prior Art

Chronic subdural hematomas (CSDHs) are debilitating conditions and impact significant percentages of the population, particularly the elderly, both within the United States and worldwide. CSDHs involve blood collecting between the arachnoid layer and the dural layer of the brain surface, and can be caused by disruptions of veins, arteries, and capillary networks. These disruptions can result from traumatic incidents, as well as from the use of anti-platelet and anticoagulant medications; moreover, CSDHs can cause weakness, language deficits, seizures, impaired consciousness, and death. The current worldwide annual incidence of CSDHs ranges from 1-5 occurrences per 100,000, but CSDHs disproportionally affect elderly populations, with annual incidence rates in those over 70 years of age being as high as 58 occurrences per 100,000. Moreover, IPHs represent the most common form of hemorrhagic stroke, occurring at a rate of 24.6 per 100,000 person-years. By 2030, 19% of the United States population is projected to be over the age of 65, thereby increasing the likely volume of persons affected by CSDHs. Between 1998 and 2007, annual hospitalization rates for treating subdural hematomas via hospitalizations increased from 39-per-100,000 (per capita) to 41.6-per-100,000 (per capita), and the current estimated cost of such hospitalization is $1.6 billion annually. CSDHs are projected to be the most common condition requiring neurosurgical intervention by 2030.

Treatment of subdural hematomas is typically performed by creating a burr hole within a patient's head and attaching a port to the patient's skull. However, these ports often have small internal lumens, e.g., 5 mm or less. These smaller lumens are prone to clogging and typically result in the failure to completely remove the hematoma due to the small diameter of the internal lumens. As such, the recurrence rate of the CSDHs can be as high as approximately 28%.

Until the present invention, it appeared impossible to secure larger ports in the minimal bone depth available in the skull due to imprecise burr holes. Attempts were made, but the larger ports suffered from poor pull-out strength that led to unintended removal of the ports.

Accordingly, what is needed is a surgical instrument to condition large diameter burr holes to enable installation of a large diameter port and improve procedural outcomes. However, in view of the art considered as a whole at the time the present invention was made, it was not obvious to those of ordinary skill in the field of this invention how the shortcomings of the prior art could be overcome.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for a surgical instrument and method to create large diameter burr holes to enable installation of a large diameter port and improve surgical procedural outcomes is now met by a new, useful, and nonobvious invention.

The present invention includes a device and method for creating large diameter burr holes to enable installation of a large diameter port within a patient. In some embodiments, the device includes a precision cutting tool having a proximal end opposite a distal end, with a body extending between the proximal end and the distal end. The proximal end is secured to or attachable to a rotatable connection on a drill while a cutting structure is proximate the distal end.

The cutting structure includes a plurality of radial cutting edges and a plurality of nose cutting edges. Each of the plurality of radial cutting edges extending outwardly in a radial direction and each of the plurality of nose cutting edges extending outwardly in a distal direction at the distal end of the precision cutting tool. In some embodiments, the plurality of radial cutting edges includes five or more radial cutting edges and/or the plurality of nose cutting edges includes five or more nose cutting edges. The precision cutting tool may further include a plurality of helical flutes with each of the plurality of helical flutes connected to one of the plurality of radial cutting edges and/or one of the plurality of nose cutting edges.

In some embodiments, each of the radial cutting edges of the precision cutting tool includes a rake angle between approximately −20°and approximately 20°, at least one secondary rake angle on diameter between approximately 3° and approximately 30°, and/or a phase width surface with an arc length between approximately 0.1 mm and approximately 0.6 mm. In addition, each of the nose cutting edges of the precision cutting tool includes a rake angle between approximately −20° and approximately 20°, at least one relief surface having a relief angle between approximately 3° and approximately 30°, and at least one clearance surface with a clearance angle between 3° and approximately 30°.

The cutting structure may further include a centrally recessed region disposed in the distal end of the surgical instrument and/or an outer diameter greater than or equal to approximately 7 mm.

Some embodiments of the precision cutting tool include a safety stop a predetermined distance from the distal end of the precision cutting tool. The safety stop may be a distal end of a removable cover when the removable cover is operably engaged to the surgical instrument. The present invention may further include a method for creating large diameter burr holes to enable installation of a large diameter port within a patient. The method includes conditioning an initial hole to create an implantation hole. The conditioning step includes rotating a precision cutting tool within the initial hole. The precision cutting tool includes a cutting structure with a plurality of radial cutting edges extending outwardly in a radial direction and each of the plurality of radial cutting edges connected to one of a plurality of helical flutes extending along the body of the precision cutting tool. The cutting structure also includes a plurality of nose cutting blades extending outwardly in a distal direction at the distal end of the precision cutting tool with each of the plurality of nose cutting blades connected to one of the plurality of helical flutes. The plurality of radial cutting edges and the plurality of nose cutting blades are configured to remove an amount of material from a bone of the subject to create the implantation hole.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is a perspective view of a precision cutting tool in accordance with an embodiment of the present invention.

FIG. 2 is a perspective view of an embodiment of the precision cutting tool with the cover removed.

FIG. 3 is a cross-sectional view of an embodiment of the precision cutting tool.

FIG. 4 is a cross-sectional close-up side view of a distal end of an embodiment of the precision cutting tool.

FIG. 5 is an end view of a distal end of an embodiment of the precision cutting tool.

FIG. 6 is a perspective end view of a portion of the distal end of an embodiment of the precision cutting tool.

FIG. 7 is a cross-sectional view of a distal end of an embodiment of the precision cutting tool.

FIG. 8 is a cross-sectional view of a nose cutting edge of an embodiment of the precision cutting tool.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that other embodiments may be utilized, and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

All numerical designations, such as measurements, efficacies, physical characteristics, forces, and other designations, including ranges, are approximations which are varied up or down by increments of 1.0 or 0.1, as appropriate. It is to be understood, even if it is not always explicitly stated that all numerical designations are preceded by the term “about” or “approximately.” As used herein, “about” or “approximately” refers to being within an acceptable error range for the particular value as determined by one of ordinary skill in the art, which will depend in part on how the value is measured or determined. For example, the term “approximately” can refer to ±10% of the numerical values.

As used herein, “subject” or “patient” is used to describe a human or other animal to whom treatment is administered.

As used herein, “target area” is used to describe an area of a subject that requires medical attention, such as a skull of a subject experiencing symptoms resulting from a subdural hematoma.

The present invention includes a surgical instrument and method of using said instrument to condition a large burr hole in a patient. In some instances, the conditioned burr hole allows for non-operational (such as bedside) evacuations of subdural hematomas, with a reduced recurrence rate, such that the use of general anesthesia can be avoided, and subdural hematomas can be treated with reduced risk to the impacted population. The improved surgical instrument and method of use will be described in greater detail in the sections below. It should be noted that while the present invention will be described herein in relation to cranial procedures, the system, its components, and the method of use can be used on other anatomy of a patient and/or to perform other procedures, including non-surgical procedures.

As previously explained, installing a port in an implantation hole larger than 5 mm was error prone and the port was failing to remain securely in the bone. A contributing reason to these issues was that the skull has a minimal thickness, ranging from approximately 4.7 mm to approximately 14.7 mm with a mean thickness of approximately 8 6 mm. As a result of the minimal thickness, it was determined that an extremely precise hole was required to securely implant a port in a skull. More specifically, the implantation hole requires a previously unachieved level of precision in relation to its perpendicular orientation with the implant site, the conditioning of the internal walls, and a uniform internal diameter. Thus, the present invention includes a precision cutting tool designed to achieve these requirements.

Referring now to the Figures, an embodiment of the present invention includes a precision cutting tool 100. In some embodiments, the precision cutting tool 100 is configured to condition an initial hole created by an initial cutting tool, thereby creating an implantation hole.

The precision cutting tool 100 is comprised of a material sufficiently hard to cut bone and tissue. In some embodiments, precision cutting tool 100 is made of a hard metal such as stainless steel, carbon steel, titanium, tungsten carbon, combinations thereof, or similar materials of similar hardness.

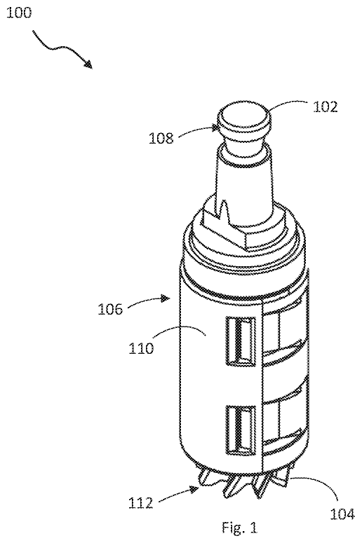

As depicted in FIGS. 1-3, precision cutting tool 100 includes a proximal end 102, a distal cutting end 104, and a body section 106 extending therebetween. The proximal end 102 is configured to be received by a connection receptacle of a drill or may be permanently secured to the rotational component of a drill. When configured to be received by a connection receptacle of a drill, the proximal end 102 is designed with a fitting 108, such as the depicted Hudson fitting, that is selectively receivable and securable within the connection receptacle of a drill.

Some embodiments include a cover 110 at least partially surrounding the precision cutting tool 100. Cover 110 has a length less than the length of the precision cutting tool 100, such that the proximal end 102 can be received by the connection receptacle of a drill and a portion 112 of the distal cutting end 104 remains exposed for engaging and cutting bone. The distal most end 114 of cover 110 acts as a safety stop to prevent further penetration of the precision cutting tool 100 beyond the length of the exposed distal end 112. Some embodiments alternatively rely on a shelf disposed between the proximal end 102 and the distal end 104 of the precision cutting tool 100 to function as a safety stop. In another embodiment, a collar is disposed between the proximal end 102 and the distal end 104 of the precision cutting tool 100. It should be appreciated that any stopping component, such as a terminal edge, a shelf, a collar, and a similar mechanical component can be used in combination with the precision cutting tool 100, so long as the stopping component includes a diameter that is greater than a diameter of the exposed portion 112 of the precision cutting tool 100.

Moreover, the cover 110 can be attached to the precision cutting tool 100 in such a manner that the cover 110 can spin independently of the precision cutting tool 100. As a result, the cover 110 can remain stationary while the precision cutting tool 100 rotates. This prevents damage to the skin and hair of the patient during use.

In an embodiment, the exposed portion 112 is approximately 5 mm in length and requires a target area (such as a skull) having a thickness of at least 6 mm to avoid complete penetration of the inner table of the skull; however, it should be appreciated that varying lengths of the exposed portion 112 can be used with varying bone thicknesses. For example, in an embodiment, sufficient engagement can be accomplished with approximately 2 mm of depth; as such, in an embodiment, the exposed portion 112 is approximately 2 mm in length. Other lengths of the exposed portion 112, including 3 mm, 3.5 mm, 4 mm, and 4.5 mm, are contemplated herein to accomplish sufficient engagement with a target area of a subject. In some embodiments, the length of the exposed portion 112 is between approximately 2 mm and 6 mm. In some embodiments, the length of the exposed portion 112 is between approximately 2 mm and 14 mm.

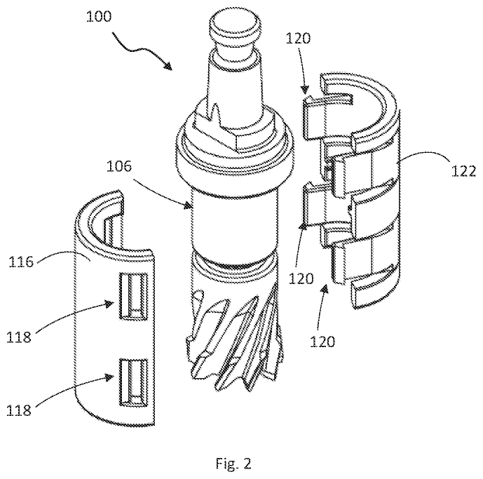

To achieve alternative lengths of the exposed portion 112, some embodiments include a plurality of covers having different lengths. Moreover, the one or more covers 110 can be detachable as best depicted in FIG. 2. In such embodiments, the cover 110 includes one or more sections that are detachable from each other through a mechanical, magnetic, or other attachment mechanism. The depicted embodiment of the cover 110 includes two sides with a first side 116 having one or more receipts 118 sized to receive the cantilevered snaps 120 on a second side 122. The cantilever snaps 120 can flex under force to enter and exit the receipts 118. Again, alternative mechanisms can be employed to secure one or more sections of the cover 110 around the body section 106.

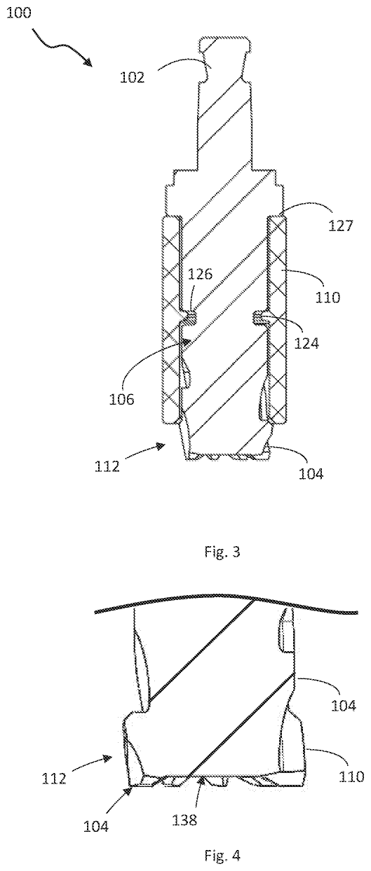

As best depicted in FIG. 3 the one or more covers 110 include one or more inwardly extending projections 124. The body section 106 of the precision cutting tool 100 includes a projection receipt 126 configured to receive the projections 124 to help retain the cover 110 at a location relative to the distal cutting end 104 of the precision cutting tool 100. The depicted receipt 126 is in the form of an annular groove to receive the annular projection 124. However, alternative retention structures are considered, including but not limited to discrete semicircular projections and receipts.

Some embodiments of the precision cutting tool 100 further include a shoulder 127 configured to contact a proximal end of the cover 110, thereby preventing the cover 110 from translating proximally relative to the body section 106. Like the projection receipt 126, the shoulder 127 ensures that the cover 110 provides the necessary safety stop during use.

Referring now to FIGS. 4-8, the exposed portion 112 (also referred to as “the cutting structure”) of the precision cutting tool 100 includes a plurality of radial cutting edges 130 and a plurality of helical flutes 128 that are proximate to the distal end 104 of the precision cutting tool 100, such that upon insertion into the initial hole, the plurality of radial cutting edges 130 are configured to interact with the internal side wall that defines the initial hole. The radial cutting edges 130 are arranged in a generally parallel orientation relative to the central rotational axis of the precision cutting tool 100. Thus, as the precision cutting tool 100 rotates, the radial cutting edges 130 remove material from the side walls of the initial hole upon insertion therein.

Rather than utilizing flutes that are substantially parallel to the internal side walls that define the initial hole, or flutes that are substantially perpendicular to the internal side walls that define the initial hole, the plurality of flutes 128 of the precision cutting tool 100 extend in a generally helical manner about a circumference of at least the cutting structure of the precision cutting tool 100. The radial cutting edges 130 and helical flutes 128 are such that the precision cutting tool 100 is configured to remove a minimal amount of material in a radial direction within the initial hole, such that the flutes 128 and the radial cutting edges 130 function similar to a reaming tool to condition the diameter of the hole to a very precise and uniform diameter.

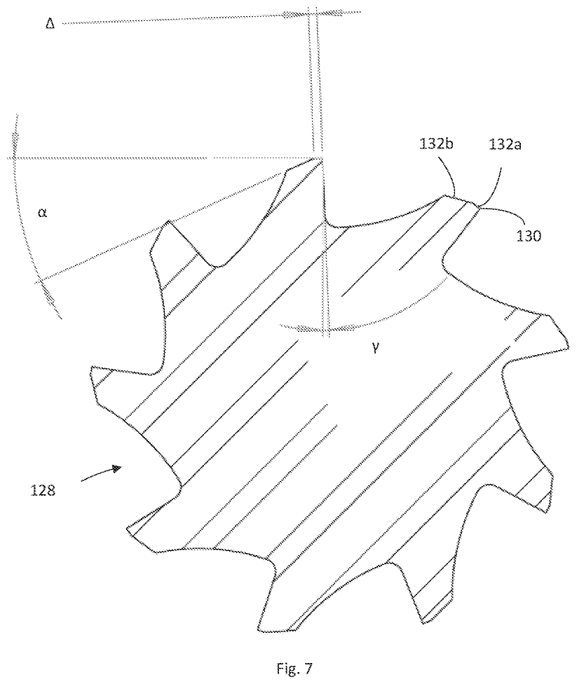

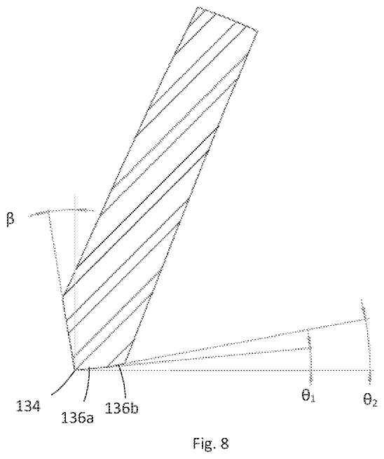

As best depicted in FIG. 7, the plurality of radial cutting edges 130 include a rake angle y of approximately 2°, however the rake angle y can be between approximately −20° and 20°. To ensure that the radial cutting edges 130 are sufficiently strong and create minimal debris during cutting, the plurality of radial cutting edges 130 also include one or more relief surfaces 132 provided on the burr surface trailing each of the cutting edges 130. Some embodiments include a phase width surface 132a and a secondary relief surface 132b in different planes as depicted. The phase width surface 132 a is depicted as having an arc length Δ of approximately 0.24 mm and the secondary relief surface 132b is depicted as having a secondary rake angle on diameter α of approximately 24°, however, the phase width can have an arc length between approximately 0.1 mm and 0.6 mm and the secondary rake angle on diameter can be between approximately 3°and 30°.

The combination of the rake and/or relief angles ensures that the plurality of radial cutting edges 130 precisely condition the initial hole and minimally increase the diameter of the initial hole through the removal of the minimal amount of material. In addition, these characteristics allow the plurality of radial cutting edges 130 to create a substantially uniform diameter from an insertion end of the initial hole to a terminal end of the initial hole.

To establish a uniform diameter, the cutting structure has an outer diameter as established by the radial cutting edges 130. The outer diameter of the cutting structure is larger than the outer diameter of the initial cutting tool and/or the initial hole. In some embodiments, the outer diameter of the cutting structure is between approximately 7 mm and 16 mm. In some embodiments, the outer diameter of the cutting structure is at least 7 mm. In some embodiments, the outer diameter of the cutting structure is roughly 14 mm.

The larger outer diameters disclosed above in relation to the cutting depths (i.e., the length of the cutting structure from the safety stop to the distal end), establishes a large a diameter-to-depth ratio in which the diameter is greater than or equal to the length of the cutting structure from the safety stop to the distal end. For example, when the length of the cutting structure from the safety stop to the distal end is 3 mm, non-limiting examples of the diameter-to-depth ratio include 6:3, 7:3, 14:3, 15:3 and even up to 20:3. As another example, when the length of the cutting structure from the safety stop to the distal end is 5 mm, the diameter-to-depth ratio can be 6:5, 7:5, 14:5, 15:5, and even 20:5.

The cutting structure of the precision cutting tool 100 also includes a plurality of nose cutting edges 134 that are disposed at the distal end 104 of the precision cutting tool 100, such that each of the plurality of flutes 128 terminates at an individual respective nose cutting edge 134. As best depicted in FIG. 7, each of the plurality of nose cutting edges 134 includes a rake angle β and relief/clearance angle(s) θ to create a cutting surface that is similar to an endmill tool. In an embodiment, these angles of the plurality of nose cutting edges 134 are greater than an angle of the helical orientation of the plurality of flutes 128; however, it should be appreciated that varying angles of the plurality of nose cutting edges 134 and the plurality of flutes 128 are contemplated herein, so long as sufficient depth can be achieved in the initial hole when modified by the precision cutting tool 100. As depicted in FIG. 7, the rake angle β of the plurality of nose cutting edges 134 is approximately −9° and the relief angle θ₁ and the clearance angle θ₂ of the plurality of nose cutting edges 134 are approximately 5° and 10°, respectively. In an embodiment, the rake angle β of the plurality of nose cutting edges 134 is between approximately −20° and 20°, and the relief angle θ₁ and the clearance angle θ₂ of the plurality of nose cutting edges 134 are between approximately 3° and 45°.

To ensure that the nose cutting edges 134 are sufficiently strong and create minimal debris during cutting, one or more relief surfaces are provided on the burr surfaces trailing the cutting edges 134. Some embodiments include at least one relief surface 136a and at least one clearance surface 136b in different planes, with corresponding relief angle θ₁ between approximately 3° and 30° and the clearance angle θ₂ between approximately 3° and 30°. Some embodiments include 3 or more relief surfaces in different planes.

The rake and relief/clearance angles of the plurality of nose cutting edges 134 together create a cutting surface that provides for the removal of material in the axial direction toward the terminal end of the initial hole, such as the subject's skull surface. Together, the plurality of radial cutting edges 130 and the plurality of nose cutting edges 134 remove minimal material radially and axially to precisely condition the diameter and depth of the initial hole to receive and secure a port therein. This results in the modification of the initial hole into an implantation hole that provides, for example, intracranial access with sufficient depth to ensure that a port inserted therein is not prone to unintentional dislodgement. Moreover, the helical nature of the plurality of flutes 128 is such that the material removed by the plurality of radial cutting edges 130 and by the plurality of nose cutting edges 134 is removed from the implantation hole, with the removed material being pushed away from the terminal end as more material is removed and traveling through the helixes of the plurality of flutes 128.

The distal end 104 of precision cutting tool 100 further includes a centrally recessed region 138 relative to the distal projections of the nose cutting edges 134. Because the recessed region 138 is counter-sunk, the distal end 104 has sufficient space to receive bone chips when cut from the hole and is also less susceptible to plunging into the patient's tissue.

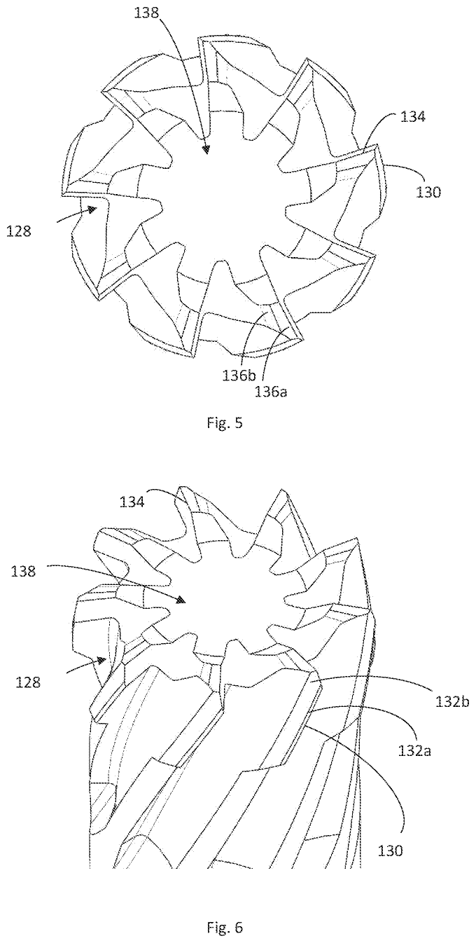

As best depicted in FIGS. 5-7, precision cutting tool 100 includes nine nose cutting edges 134 and nine radial cutting edges 130 leading to nine flutes 128 to create a precise implantation hole. Some embodiments may have five or more nose cutting edges 134 and five or more radial cutting edges 130 leading to five or more respective flutes 128 to achieve an implantation hole precise enough to securely receive a port. In some embodiments, the number of flutes 128 corresponds with the number of nose cutting edges 134 and/or the number of radial cutting edges 130.

The present invention further includes a method of conditioning a large diameter burr hole within a patient. The method includes conditioning an initial hole in the patient at a target site, e.g., the head of the patient, into an implantation hole with a precise diameter. The conditioning step includes rotating a precision cutting tool within the initial hole. The precision cutting tool may be of a design in accordance with the precision cutting tool 100 as described herein.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

1. A surgical instrument for conditioning a large diameter burr hole in a patient, comprising:
a proximal end opposite a distal end, with a body extending between the proximal end and the distal end;
a cutting structure proximate the distal end, the cutting structure including:
a plurality of radial cutting edges, each of the plurality of radial cutting edges extending outwardly in a radial direction;
a plurality of nose cutting edges, each of the plurality of nose cutting edges extending outwardly in a distal direction at the distal end of the surgical instrument;
an outer diameter greater than or equal to approximately 7 mm.

2. The surgical instrument of claim 1, further including a centrally recessed region disposed in the distal end of the surgical instrument.

3. The surgical instrument of claim 1, wherein the plurality of radial cutting edges and plurality of nose cutting edges includes five or more radial cutting edges.

4. (canceled)

5. The surgical instrument of claim 1, further including a plurality of helical flutes, each of the plurality of helical flutes connected to one of the plurality of radial cutting edges or one of the plurality of nose cutting edges.

6. (canceled)

7. The surgical instrument of claim 1, wherein each of the radial cutting edges includes a rake angle between approximately −20° and approximately 20°.

8. The surgical instrument of claim 1, wherein each of the radial cutting edges includes a relief angle between approximately 3° and approximately 30°.

9. The surgical instrument of claim 1, wherein each of the nose cutting edges includes a rake angle between approximately −20° and approximately 20°.

10. The surgical instrument of claim 1, wherein each of the nose cutting edges includes at least one relief surface having a relief angle between approximately 3° and approximately 45°.

11. The surgical instrument of claim 1, further including a safety stop at a predetermined location from the distal end of the precision cutting tool thereby establishing a maximum penetration depth of the precision cutting tool.

12-13. (canceled)

14. A precision cutting tool for creating a large diameter burr hole in a patient, comprising:
a proximal end opposite a distal end, with a body extending from between the proximal end and the distal end;
a cutting structure, the cutting structure including:
a plurality of helical flutes extending along at least a portion of the body of the precision cutting too
a plurality of radial cutting edges extending outwardly in a radial direction, each of the plurality of radial cutting edges connected to one of the plurality of helical flutes;
a plurality of nose cutting blades extending outwardly in a distal direction at the distal end of the precision cutting tool, each of the plurality of nose cutting blades connected to one of the plurality of helical flutes;
wherein the plurality of radial cutting edges and the plurality of nose cutting blades are configured to remove an amount of material from a bone of the patient to create an implantation hole.

15. The precision cutting tool of claim 14, wherein the precision cutting tool includes at least five radial cutting edges and at least five helical flutes.

16. The precision cutting tool of claim 14, further including a centrally recessed region disposed in the distal end of the precision cutting tool.

17. The precision cutting tool of claim 14, wherein the plurality of radial cutting edges includes five or more radial cutting edges.

18. The precision cutting tool of claim 14, wherein the plurality of nose cutting edges includes five or more nose cutting edges.

19. The precision cutting tool of claim 14, further including a plurality of helical flutes, each of the plurality of helical flutes connected one of the plurality of radial cutting edges.

20. The precision cutting tool of claim 14, further including a plurality of helical flutes, each of the plurality of helical flutes connected one of the plurality of nose cutting edges.

21-25. (canceled)

26. The precision cutting tool of claim 14, further including a safety stop at a predetermined location from the distal end of the precision cutting tool thereby establishing a maximum penetration depth of the precision cutting tool.

27. The precision cutting tool of claim 14, further including a safety stop, wherein the safety stop is a distal end of a removable cover when the removable cover is securely attached to the precision cutting tool.

28. The precision cutting tool of claim 14, wherein the proximal end is secured to or attachable to a rotatable connection on a drill.

29. A method of conditioning a large diameter burr hole in a patient, the method comprising:
conditioning an initial hole in the patient to create an implantation hole, wherein conditioning includes rotating a precision cutting tool within the initial hole, the precision cutting tool including:
a proximal end opposite a distal end, with a body extending from between the proximal end and the distal end;
a cutting structure, the cutting structure including:
a plurality of helical flutes extending along at least portion of the body;
a plurality of radial cutting edges extending outwardly in a radial direction, each of the plurality of radial cutting edges connected to one of the plurality of helical flutes;
a plurality of nose cutting edges extending outwardly in a distal direction at the distal end of the precision cutting tool, each of the plurality of nose cutting edges connected to one of the plurality of helical flutes;
wherein the plurality of radial cutting edges and the plurality of nose cutting edges are configured to remove an amount of material from a bone of the patient to create the implantation hole.

30-43. (canceled)
